(19) United States
(12) Patent Application Publication
(10) Pub. No.: US 2010/0004560 A1
(43) Pub. Date: Jan. 7, 2010

(54) CONFUSER CROWN SKIN PRICKER

(75) Inventors: GLENN DAVISON, Oxford (GB); Jeremy Michael John Marshall, Oxford (GB)

Correspondence Address: JOHN S. PRATT, ESQ;KILPATRICK STOCKTON, LLP, 1100 PEACHTREE STREET, SUITE 2800, ATLANTA, GA, 30309, US

(73) Assignee: OWEN MUMFORD, LTD., Oxfordshire (GB)

(21) Appl. No.: 12/557,833

(22) Filed: Sep. 11, 2009

Related U.S. Application Data
(63) Continuation of application No. 11/712,754, filed on Mar. 1, 2007, now abandoned.
Application No. 11/712,754 is a continuation of application No. 10/635,806, filed on Aug. 5, 2003, now abandoned.
Application No. 10/635,806 is a continuation-in-part of application No. 09/959,262, filed on Oct. 22, 2001, now abandoned.

(51) Int. Cl. A61B 5/151; A61B 17/34

(52) U.S. Cl. 600/583; 606/181

(57) ABSTRACT

A blood sampling device designed to divert attention from the pain involved in the puncture of the skin tissue by increasing comfort when lancing the skin by affecting the sensation and perception of pain. Projections on the distal end of a skin pricker contact the skin surface to confuse the nerves in the area of the prick to make the prick less noticeable.

### RELATED APPLICATION DATA

This application is a continuation-in-part of U.S. application Ser. No. 09/959,262, filed Oct. 22, 2001, which is incorporated herein by reference in its entirety.

### FIELD OF THE INVENTION

This invention relates generally to medical skin piercing devices including blood sampling devices, and more particularly to skin prickers.

### BACKGROUND

Blood sampling devices are used to draw a small drop of blood for analysis. One type of blood sampling device is a skin pricker. Such skin prickers are widely used by diabetics, for example, who need to know their sugar level. However, there are many other applications.

There are many different types of skin prickers, including spring-loaded devices that fire disposable lancets. Some pricking devices are entirely disposable after a single use. Regardless of the form, the pricker punctures the skin of the user and inflicts pain. While this pain is somewhat trivial and transitory, many users would welcome its reduction.

### SUMMARY

This invention is a skin pricker designed to divert attention from the pain involved in the puncture of the skin tissue by a blood sampling device. The skin pricker of this invention increases comfort when lancing by affecting the sensation and perception of pain. Projections on the distal end of a lancing device, or skin pricker, contact the skin surface to confuse the nerves in the area of the prick to make the prick less noticeable. This approach disguises the lancing action to provide a more comfortable sample.

### BRIEF DESCRIPTION OF THE DRAWINGS

FIG. 1 is a perspective view of a skin pricker of this invention.

FIG. 2 is an enlarged perspective view of the depth adjuster of the skin pricker of FIG. 1.

FIG. 3 is an exploded perspective view of the skin pricker of FIG. 1.

FIG. 4 is a side elevation view in cross-section of the skin pricker of FIG. 1.

FIG. 5 is a side elevation view of the skin pricker of FIG. 1.

FIG. 6 is an end view of the skin pricker of FIG. 1.

FIG. 7 is a top plan view of the skin pricker of FIG. 1.

### DETAILED DESCRIPTION

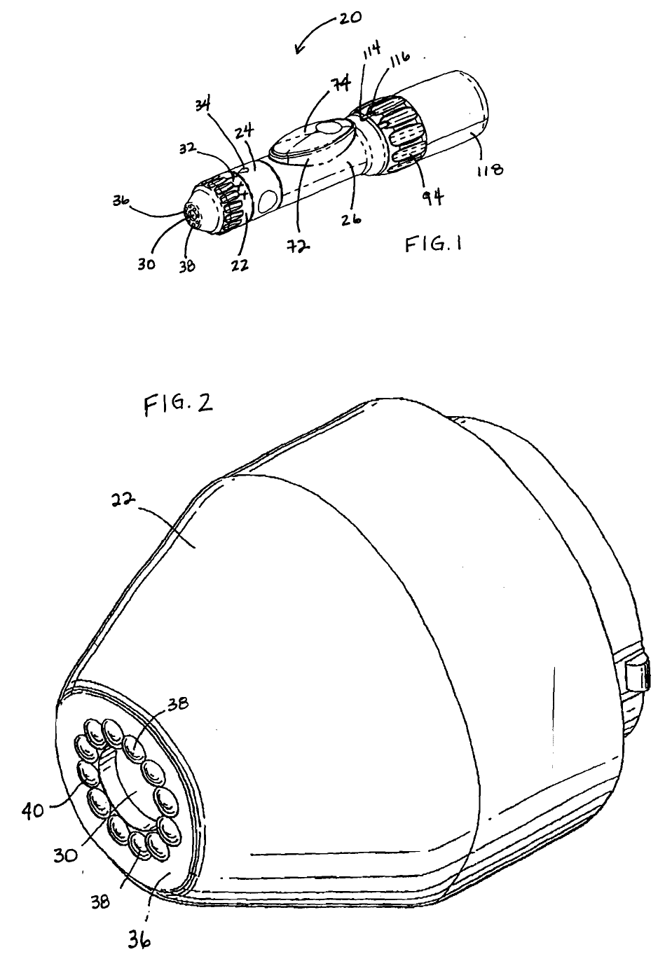

A skin pricker of this invention is a lancing device 20 for firing a lancet, as shown in the Figures. As may be seen by reference to FIGS. 1 and 2, lancing device 20 includes depth adjuster 22, which includes interior threads (not shown) adapted to engage a front clip 24, which engages a body 26. In the description below, distal refers to the nose or lancing end generally, while proximal indicates a direction away from the nose of the device.

Lancing device 20 may be used with a suitable disposable lancet, which may be inserted into lancet holder 28 by removing the assembly of the depth adjuster 22 and front clip 24. Dialing depth adjuster 22 relative to front clip 24 alters the distance between the front clip 24 and the depth adjuster 22, but does not disrupt engagement between the clip 24 and adjuster 22. Therefore dialing of the adjuster 22 controls how far the lancet tip will project beyond aperture 30 of adjuster 22. In the embodiment shown, depth adjuster 22 includes indicia 32 that correspond to penetration depth indicator 34, which together reflect the expected penetration level of the lancet into the skin of the patient.

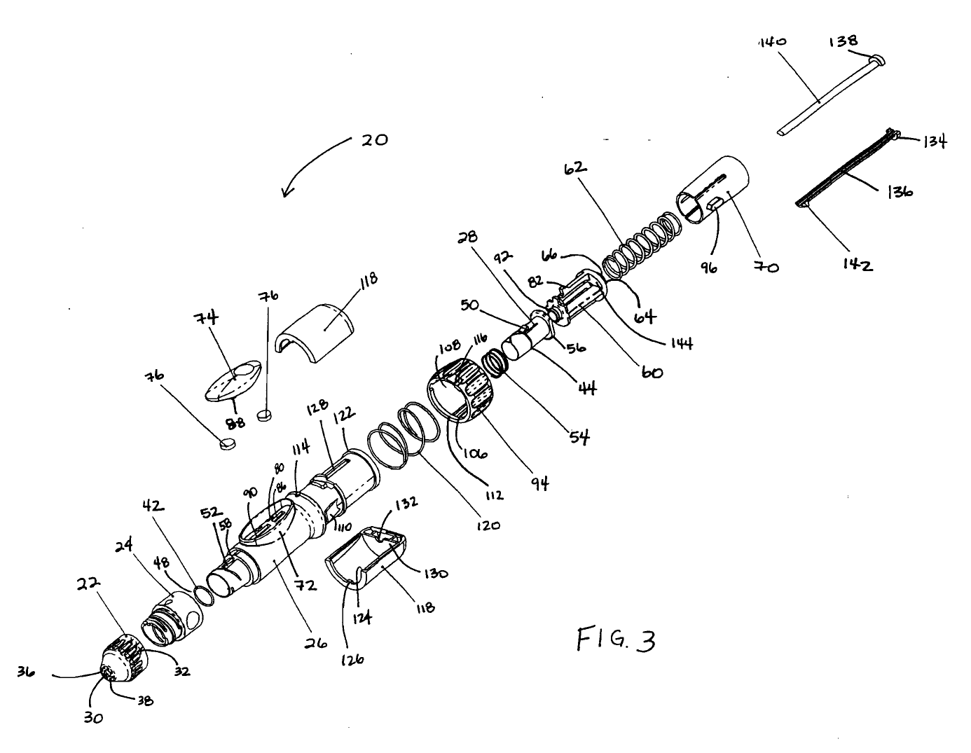

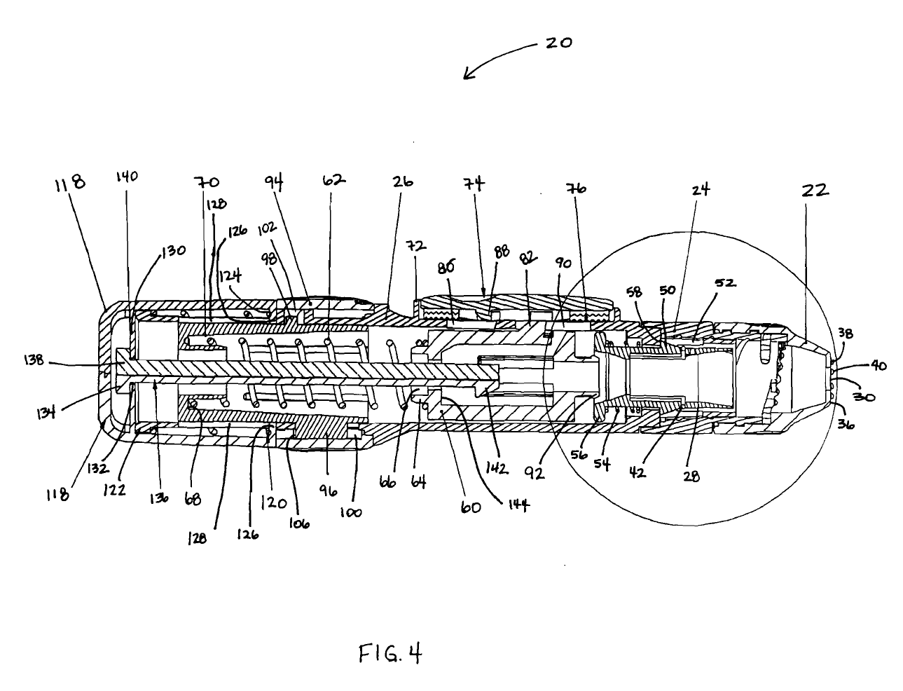

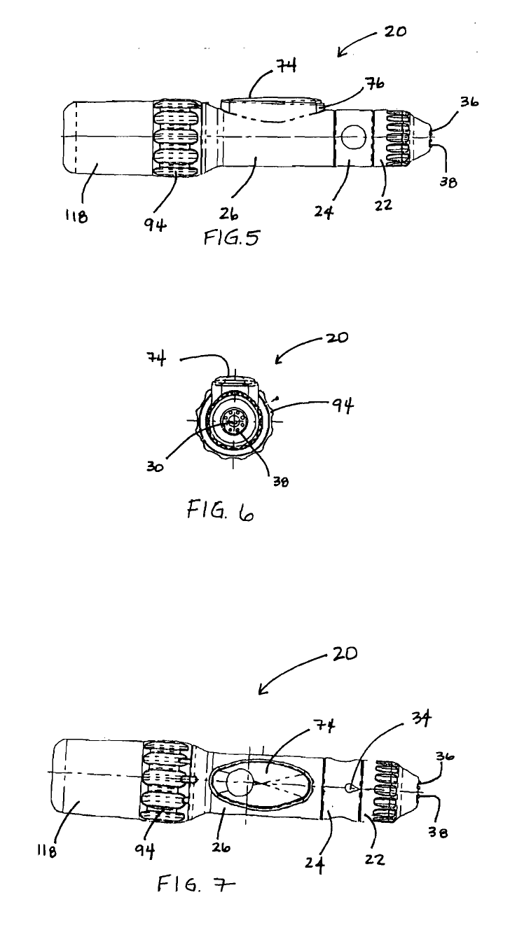

As may be shown by reference to FIGS. 3 and 4, depth adjuster 22 includes platform 36 and aperture 30. Projections 38 encircle aperture 30, and each projection 38 has a shallow conical form with a rounded tip 40. The number and arrangement of the projections 38 may vary, as may the size and shape of the individual projections. For example, the arrangement may zigzag as it surrounds the aperture. Pyramidal projections may be used, or any other suitable shape. Alternatively, two rows of projections may encircle the aperture. The two rows may include projections having various shapes and may spaced at various distances. Projections having various shapes may also be used on the same device. The projections contact the skin surface to confuse the nerves in the area of the prick, making the prick less noticeable.

As shown in FIGS. 2 and 4, lancet retention clip 42 fits into groove 44 on lancet holder 28, retaining a lancet in lancet holder 28. Clip 42 includes slit 48, which allows clip 42 to expand for installation. Wings 50 of lancet holder 28 are adapted to be received in corresponding wing slots 52 on the distal portion of body 26.

Recoil spring 54 fits around lancet holder 28 between cap 56 and wings 50, and urges lancet holder 28 in a proximal direction, or rearward, in a resting state where the lancet does not project beyond the nose. The recoil spring 54 urges the lancet back to this resting state immediately after firing and lancing. Wings 50 translate in slots 52, and catch on wing stops 58, preventing the lancet holder 28 from being recoiled beyond the distal portion of the body 26 after firing of the lancet.

Hammer 60 is urged toward the distal end of body 26 by main spring 62. The distal end of the main spring 62 contacts the proximal end of the hammer 60, fitting around ring 64 that projects from the proximal end surface of the hammer 60 and that surrounds the aperture 66 of the hammer 60. The proximal end of main spring 62 rests against inner end surface 68 of the force adjuster 70.

Body 26 includes button frame 72 adapted to receive firing button 74. Foam pads 76 on the interior of firing button 74 contact the outer surface of body 26, and clips (not visible) on the interior of the button 74 are received in holes (not visible) in the body 26, securing the button 74 to the body 26 in the frame 72. In a loaded position, finger 82 of hammer 60 translates in loading slot 86 of body 26. Depressing firing button 74 causes button tab 88 to depress finger 82 of hammer 60, so that main spring 62 urges hammer 60 forward, forcing finger 82 out of loading slot 86 and into firing slot 90 of body 26. Knob 92 on the distal end of hammer 60 is adapted to fit into the proximal end of lancet holder 28, so that the main spring 62 pushes the hammer 60 into contact with lancet holder 28, expelling the lancet beyond the platform 36 of the depth adjuster 22 and into contact with the patient. In this manner, hammer 60 is projected toward lancet holder 28, firing the lancet and pricking the patient.

Force adjuster ring 94 fits around body 26 proximal to the button 74. Force adjuster 70 includes longitudinal flange 96 and tab 98. Flange 96 is adapted to translate in flange opening 100 of body 26, while tab 98 translates in tab slot 102 of body 26. Flange opening 100 includes detents (not visible), so that flange 96 is locatable at set positions. In one embodiment, flange opening 100 subtends approximately 80 degrees of the circumference of the body 26 in a generally spiral path and three detents in the proximal edge of opening 100 can receive flange 96 alternatively to provide three different levels of force.

Flange 96 is captured between a pair of ridges 106 on the inner surface 108 of force adjuster ring 94. Extensions 110 on either side of body 26 abut annular ring 112 on the inner surface 108 of ring 94, maintaining the axial position of the ring 94. Extensions 110 have the force of leaf springs that are compressed when ring 94 is pressed into position and then spring out so that their ends abut annular ring 112 and thereby capture ring 94 and retain it on the body 26.

Rotating force adjuster ring 94 causes ridges 106 to force flange 96 distally or proximally to adjust the force of main spring 62 when the lancet device 20 is triggered. Thus, the force is adjusted by turning the ring 94, which moves the force adjuster 70 axially to adjust the compression of the main spring 62. Force adjustment indicator 114 on body 26 may be aligned with one of the notches 116 on ring 94, allowing the user may set the amount of force with which to deliver the lancet.

Body sleeve 118 includes two semi-cylindrical halves, which fit around proximal end of body 26, over body spring 120, which extends between cap 122 of body 26 and internal lip 124 of sleeve 118. Keys 126 of body sleeve 118 are received in and move axially along keyways 128 of body 26. Body spring 120 urges sleeve 118 distally in a resting, or loading, state.

Sleeve 118 includes shelf 130 having an aperture 132 adapted to receive the cap 134 of the loading rod 136 and the cap 138 of the support rod 140. Loading rod 136 and support rod 140 each include a flat edge that abut each other, so that the two rods extend through the force adjuster 70 and the ends of the rods are received in the proximal end of hammer 60. The use of the two part rod structure, rods 136 and 140, permit insertion of first rod 136 through the opening 66 of hammer 60 and then rod 140, so that combined rods 136 and 140 substantially fill opening 66 of hammer 60. Loading rod 136 also includes a hook 142 that engages ledge 144 of hammer 60, preventing removal.

After firing, pulling sleeve 118 in the proximal direction compresses body spring 120 and draws the assembly of the sleeve 118, the rods 136, 140 and the hammer 60 rearward, or distally, resetting finger 82 of hammer 60 into loading slot 86. The lancet may then be removed, and a new lancet inserted, by removing the assembly of the depth adjuster 22 and front clip 24.

All variations of the structures illustrated in the drawings and the materials described above are within the scope and spirit of this invention and the following claims.

1-18. (canceled)

19. A blood sampling device, comprising:
(a) a body portion having a bore and adapted to house a lancet,
(b) means for forcing the lancet through the bore in the body;
(c) a platform having an aperture, the aperture adapted to receive at least a sharp tip of the lancet and comprising a generally flat surface having an inner boundary and an outer boundary; and
(d) projections extending from the flat surface between the inner boundary and the outer boundary and surrounding the aperture to contact skin tissue.

20. The blood sampling device of claim 19, wherein the projections further comprise a pyramidal shape.

21. The blood sampling device of claim 19, wherein the projections encircle the aperture.

22. The blood sampling device of claim 19, wherein the projections further comprise a rounded tip.

23. The blood sampling device of claim 19, further comprising means for retracting the lancet into the body after the lancet is fired.

24. The blood sampling device of claim 19, further comprising a button for firing the lancet.

25. The blood sampling device of claim 19, further comprising means for adjusting the penetration depth of the lancet.

26. The blood sampling device of claim 19, wherein the means for forcing the lancet through the bore in the body comprises a spring.

27. A lancing device for obtaining a blood sample, comprising:
(a) a body portion having a bore and adapted to house a lancet; and
(b) a nose portion having a frustoconical shape and comprising
(1) an aperture adapted to receive at least a sharp tip of the lancet,
(2) a platform surrounding the aperture, the platform comprising a generally flat surface having an inner boundary and an outer boundary, and
(3) a plurality of projections extending from the flat surface between the inner boundary and the outer boundary and near the aperture to contact skin tissue.

28. The lancing device of claim 27, wherein the projections further comprise a pyramidal shape.

29. The lancing device of claim 27, wherein the projections encircle the aperture.

30. The lancing device of claim 27, wherein the projections further comprise a rounded tip.

31. The lancing device of claim 27, further comprising means for forcing the lancet through the bore in the body.

32. The lancing device of claim 27 further comprising means for retracting the lancet into the body after the lancet is fired.

33. A lancing device, comprising:
(a) a body portion having a bore and adapted to house a lancet,
(b) means for forcing the lancet through the bore in the body;
(c) an aperture adapted to receive at least a sharp tip of the lancet;
(d) a platform surrounding the aperture and comprising a generally flat surface having an inner boundary and an outer boundary;
(e) projections extending from the platform between the inner and outer boundaries of the platform; and
(f) means for retracting the lancet into the body after firing the device.

34. The lancing device of claim 33, wherein the projections further comprise a pyramidal shape.

35. The lancing device of claim 33, wherein the projections encircle the aperture.

36. The lancing device of claim 33, wherein the projections further comprise a rounded tip.
